(19) United States
(12) Patent Application Publication
(10) Pub. No.: US 2023/0084687 A1
(43) Pub. Date: Mar. 16, 2023

(54) SYSTEM AND METHOD TO SELECT PHAGE THERAPY BASED ON TIME AND LOCATION

(71) Applicant: Adaptive Phage Therapeutics, Inc., Gaithersburg, MD (US)

(72) Inventors: Rob Cohen, Kensington, MD (US); Greg Merril, Bethesda, MD (US)

(73) Assignee: Adaptive Phage Therapeutics, Inc., Gaithersburg, MD (US)

(21) Appl. No.: 17/790,183

(22) PCT Filed: Dec. 24, 2020
(86) PCT No.: PCT/US20/67003

§ 371 Date: Jun. 30, 2022

Related U.S. Application Data
(60) Provisional application No. 62/956,729, filed on Jan. 3, 2020.

(51) Int. Cl. G16H 70/60; G16H 40/20; G16H 20/10

(52) U.S. Cl. CPC G16H 70/60; G16H 40/20; G16H 20/10

(57) ABSTRACT

A method of selecting a phage formulation is described wherein the method comprises storing bacterial infection/contamination data at one or more treatment locations in a spatio-temporal infection database, the database comprising at least one of the following data fields: (1) a clinical indication, (2) a bacteria identification, (3) a clinical outcome, (4) a phage resistance status, (5) a phage susceptibility profile, (6) an antibiotic susceptibility profile, and/or (7) a lab test result relating to any one of (1)-(6); identifying one or more phage suitable for inclusion in the phage formulation by analyzing the data fields of (1)-(7) in the database to identify to one or more infections associated with a treatment location during a historical time period based at least on one or more of a frequency of infections/contamination, a geographic clustering of infections/contamination, and/or phage usage data; and generating a selected list of phage to be included in the phage formulation. Uses of formulations are also described.

### TECHNICAL FIELD

The present disclosure relates to treatment of bacterial infections and bacterially contaminated surfaces. In a particular form the present disclosure relates to the early intervention using bacteriophage treatments.

### BACKGROUND

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Many MDR infections are hospital acquired and, in some cases, can so localized to a specific unit in a hospital. Moreover, it is believed that genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections. Methicillin resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as organisms of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial was investigated following their initial isolation early in the 20th century, and they have been used clinically as antibacterial agents in some countries with some success. Notwithstanding, phage therapy was largely abandoned in the U.S. after the discovery of penicillin, and only recently has interest in phage therapeutics been renewed.

Unlike antibiotics, which are often effective against many different organisms, a phage strain is typically only effective against a single bacterial strain. Thus successful therapeutic use of phage depends on the ability to identify and administer a phage strain or multiple phage strains that can kill or inhibit the growth of a bacterial isolate associated with an infection. Empirical laboratory techniques have been developed to screen for phage susceptibility on bacterial strains (i.e. efficacy at inhibiting bacterial growth). However these techniques are time consuming, delaying the onset of effective treatment.

For example, one approach involves taking a sample from the patient and obtaining a bacterial isolate which is then tested against multiple test phage. Whilst high throughput systems such as the Host Range Quick Test (HRQT), which is based on the use of the Omnilog microarray system, enable simultaneous testing of up to 4800 (50×96 well plates) phage-host combinations, the growth phase for each combination takes 24 hours or more. Further each growth curve must then be visually inspected to estimate the capability of a phage to lyse (kill) the bacterial isolate. This manual inspection must be performed for each host-phage combination (i.e. for each well) which adds further time. As a result, this process currently takes between 24-36 hours from collection of a sample to selection of an appropriate phage treatment which can then be provided to the patient in need of treatment.

Thus, there is a need to develop faster methods or systems to provide more rapid bacteriophage treatments, or to at least provide a more useful alternative to existing systems and methods.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the examples and defined in the appended claims.

In one aspect, a method of selecting a phage formulation is disclosed, wherein said method comprises:

(a) storing bacterial infection/contamination data in a spatio-temporal infection database, in which the data is derived from bacterial isolates from one or more treatment locations, the database comprising at least one of the following data fields: (1) a clinical indication, (2) a bacteria identification, (3) a clinical outcome, (4) a phage resistance status, (5) a phage susceptibility profile, (6) an antibiotic susceptibility profile, and/or (7) a lab test result relating to any one of (1)-(6);

(b) identifying one or more phage suitable for inclusion in the phage formulation by analyzing the data fields of (1)-(7) in the database to identify to one or more infections associated with a treatment location during a historical time period based at least on one or more of a frequency of infections/contamination, a geographic clustering of infections/contamination, and/or phage usage data;

(c) generating a selected list of one or more phage(s) to be included in the phage formulation.

In a further embodiment, the data field comprising the bacteria identification is defined by genus, species, strain, sequence, and/or NCBI tax ID.

Furthermore, the method can further comprise updating the database with additional infection-related data, and repeating the identification step for a more recent historical time period and repeating the generating step if there is a change to the one or more phage identified as suitable for inclusion in the therapeutic phage formulation. Additionally, the method can be using machine learning.

In preferred aspects, the identification of one or more phage comprises calculating a PhageScore for each phage. In further preferred aspects, the PhageScore is greater than one standard deviation from the mean.

In other preferred aspects, the method further comprises generating a phage formulation. Such phage formulations can be generated from a phage inventory management system. In preferred aspects, the phage inventory management system is updated with new phage having a PhageScore higher than one standard deviation from the mean.

Such phage formulations are also contemplated, and the use of those phage formulations generated by the methods described herein. For example, use of the phage formulations can be used to (a) treat a patient suffering from a bacterial infection; or (b) treat a surface contaminated with a bacterium

In further aspects, a computing apparatus comprising: at least one memory, and at least one processor wherein the memory comprises instructions to configure the processor to perform the methods described herein. Such non-transitory, computer program product comprising computer executable instructions for performing the method described herein are also contemplated.

### BRIEF DESCRIPTION OF DRAWINGS

Embodiments of the present disclosure will be discussed with reference to the accompanying drawings wherein:

FIG. 1 is a flowchart of a method for of treating a patient at a treatment location in need of treatment of a bacterial infection according to an embodiment;

FIG. 2 is an entity resource diagram shows a database design of a spatio-temporal infection database for storing the data according to an embodiment;

FIG. 3 is a schematic diagram of a computing apparatus according to an embodiment.

In the following description, like reference characters designate like or corresponding parts throughout the figures.

### DESCRIPTION OF EMBODIMENTS

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. A “phage formulation” means at least one phage is contained within a formulation, as well as a plurality of phages (i.e., more than one phage). As understood by one of skill in the art, the term “phage” can be used to refer to a single phage or more than one phage.

The present invention can “comprise” (open-ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organisms, e.g., any animal or human, that may be suffering from a bacterial infection, particularly an infection caused by a multiple drug-resistant bacterium.

As understood herein, a “subject in need thereof” includes any human or animal suffering from a bacterial infection, including but not limited to a multiple drug-resistant bacterial infection, a microbial infection or a polymicrobial infection. Indeed, while it is contemplated herein that the methods may be used to target a specific pathogenic species, the method can also be used against essentially all human and/or animal bacterial pathogens, including but not limited to multiple drug resistant bacterial pathogens. Thus, in a particular embodiment, by employing the methods of the present invention, one of skill in the art can design and create personalized phage cocktails against many different clinically relevant bacterial pathogens, including multiple drug-resistant (MDR) bacterial pathogens.

As understood herein, an “effective amount” of a pharmaceutical composition refers to an amount of the composition suitable to elicit a therapeutically beneficial response in the subject, e.g., eradicating a bacterial pathogen in the subject. Such response may include e.g., preventing, ameliorating, treating, inhibiting, and/or reducing one of more pathological conditions associated with a bacterial infection.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2 fold, of a value. Unless otherwise stated, the term “about” means within an acceptable error range for the particular value, such as ±1-20%, preferably ±1-10% and more preferably ±1-5%. In even further embodiments, “about” should be understood to mean +/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

The term “phage sensitive” or “sensitivity profile” means a bacterial strain that is sensitive to infection and/or killing by phage and/or in growth inhibition. In other words, phage is efficacious or effective in inhibiting growth of the bacterial strain.

The term “phage insensitive” or “phage resistant” or “phage resistance” or “resistant profile” is understood to mean a bacterial strain that is insensitive, and preferably highly insensitive to infection and/or killing by phage and/or growth inhibition. That is phage is not efficacious or ineffective in inhibiting growth of the bacterial strain.

A “therapeutically effective phage formulation”, a “phage formulation” or like terms as used herein are understood to refer to a composition comprising one or more phage which are selected by the described methods to provide a clinically beneficial treatment for a bacterial infection when administered to a subject in need thereof or used on a contaminated surface.

As used herein, the term “composition” encompasses a “phage formulation” as disclosed herein which include, but are not limited to, pharmaceutical compositions comprising one or more purified phages selected by the described methods. “Pharmaceutical compositions” are familiar to one of skill in the art and typically comprise active pharmaceutical ingredients formulated in combination with inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of skill in the art and can be found, e.g., in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water, saline, and glycerol.

As used herein, the term “estimating” encompasses a wide variety of actions. For example, “estimating” may include calculating, computing, processing, determining, deriving, investigating, looking up (e.g., looking up in a table, a database or another data structure), ascertaining and the like. Also, “estimating” may include receiving (e.g., receiving information), accessing (e.g., accessing data in a memory) and the like. Also, “estimating” may include resolving, selecting, choosing, establishing and the like.

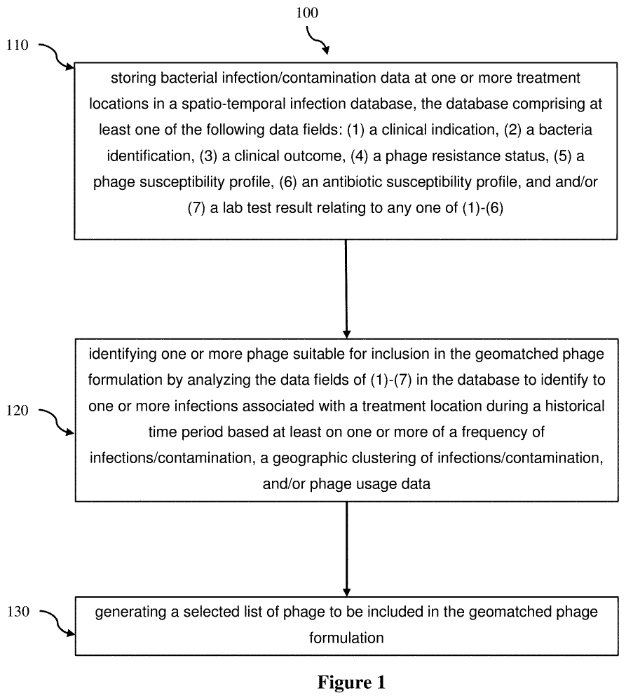

Embodiments of a method and system for treating a patient and/or a contaminated surface at a treatment location in need of such treatment will now be described. FIG. 1 is a flowchart 100 of a method and system for treating a patient/surface at a treatment location in need of treatment of a bacterial infection/contamination.

At step 110 the method begins with storing infection/contamination-related data presenting at one or more treatment locations in a database, which we will refer to as the spatio-temporal infection database. At step 120 the database is used to identify one or more phage suitable for inclusion in a phage formulation identified to be used at a treatment location. This may be performed by analysing the spatio-temporal infection database dataset to identify to one or more infections associated with a treatment location during a historical time period. This identification may be based at least on one or more of a frequency of infections/contaminations, a geographic clustering of infections/contaminations, and/or phage usage data. With suitable phage identified, the method then comprises step 130 of generating a selected list of phage to be included in a phage formulation, which can be used to (a) treat a patient presenting at the treatment location in need of treatment of an infection and/or (b) treat a contaminated surface at the treatment location. In preferred embodiments, the method allows for a hospital's phage library to be tuned to the specific infections present at a hospital, so that on presentation, a patient can be treated with a first line phage treatment that has a high probability of being effective.

For example, MDR infections tend to be acquired in hospitals. Thus if the over the last week 20 inpatients at a hospital presented with infection A, there's a good chance that patient 21 presenting with similar symptoms (or clinical indications) to the first 20 will also have infection A. As such a phage treatment appropriate to treating A could be stored on site, and immediately used to treat patient 21 upon presentation. This first line phage can thus be used whilst further testing is performed to identify the specific bacteria and/or to perform a HRQT to identify the most efficacious phage formulation for treating the patient or surface. It is noted that this could in fact be the first line phage treatment already prescribed (in which case continued treatment would be indicated). Further outcome data can then be stored to assist in selection the best phage for patient 22+.

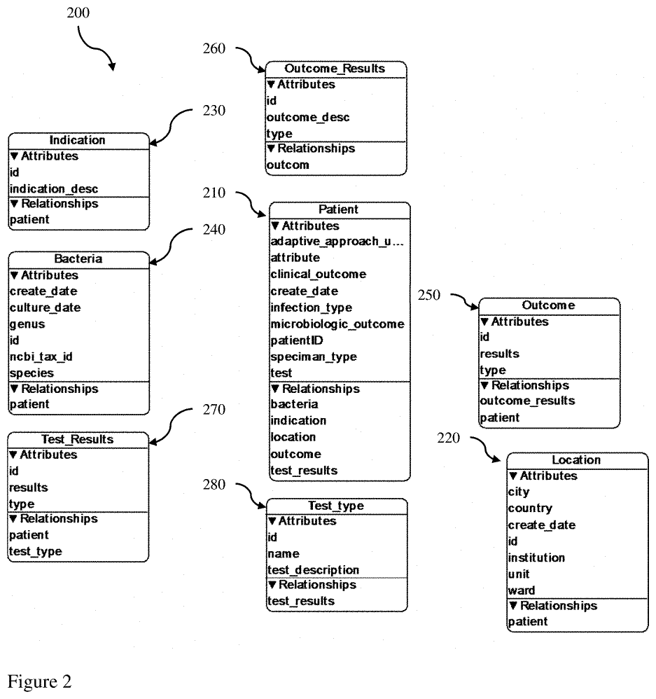

The spatio-temporal infection database may be used to store a range of infection related data which may be used to assist in identifying spatial and/or temporal trends to assist in selection of a first line phage formulation treatment for immediate treatment of a patient. FIG. 2 is an entity resource diagram shows a database design of a spatio-temporal infection database 200 for storing the data according to an embodiment. This embodiment illustrates the data items collected, such as patient data 210, location data 220, indication 230, bacteria data 240, outcome 250 and outcome results 260, test results 270 and test type 280, and the types of relationships between data items.

Location data 220 is collected to assist in identifying geographical clusters of infections. As MDR infections can be localised to specific hospitals, and even specific wards, the location data is preferable as detailed as possible. Thus, in the embodiment shown in FIG. 2, data on the city and country, as well as the institution, unit and ward are collected. In some embodiments, geographic coordinates (e.g. latitude, longitude) may be collected or obtained based on an institution name.

Patient data 210 relevant to identification of infections and outcomes is collected and stored. As shown in FIG. 2, this may include details such as a patient ID, creation date (i.e. date/time of first presentation), clinical outcome (e.g. infection resolved or not), microbiological outcome (e.g. bacteria no longer detectable), infection type, specimen type, tests performed, phage treatments administered, etc. this may originally be captured in patient medical record, and the relevant data may then be extracted and stored in a patient record of the database 200. In one embodiment a scheduled task is created to automatically query patient medical records and extract indication data for storage in the spatio-temporal infection database. Data may be anonymised during extraction to protect patient confidentiality.

Clinical indication data 230 includes observed or reported symptom by the patient to assist in identifying the bacteria, infection, or contamination. For example, if patients exhibiting similar indications, or patterns of indications, this may indicate they are suffering from an infection due to the same bacteria. A database table for indication data may store an id for the indication, and an associated description of the indication for the patient. For example indicators of infection may be temperature, cough with a description of the type of cough (dry/wet), pain, with a description of the location and nature, etc. This indication data may originally be stored in a patient's medical record, along with a time the information was captured, or the time the symptoms occurred, and this data may be extracted and stored in indication table 230 of the spatio-temporal infection database 200. Similarly, and at a higher level, clinical diagnosis (such as urinary tract infection) can also be included in the database.

Bacterial data 240 includes data identifying the bacteria isolated from a patient sample (e.g. sputum, swab, blood test, etc). For example, a patient sample may be collected along with a time of collection. The sample may be then prepared and cultured to allow bacteria present to grow. After a growth stage, and in one embodiment, colonies can be sequenced to identify the specific bacteria present. Alternative direct sequencing could be performed on sample and bioinformatics analysis methods used to identify one or more bacteria present in the sample. For example shotgun sequencing approaches such as metagenomic next generation sequencing may be used. As shown in FIG. 2, the bacterial data collected may include identification information such as genus, NCBI taxonomy ID, species, as well as details such as creation date and culture date.

Outcome data 250 is stored which may be a results code (e.g. 1=cured/resolved, 0=not cured/resolved) along with type data on a type of outcome measure (clinical outcomes, microbiological outcome). The results code may be a binary indicator, or an enumerated set of results (cured, improved, improved then relapsed, no effect). Related to outcome data 250 is outcome results 260 which provides a description of the outcome providing greater details on the nature of the outcome beyond a binary or outcome result code.

Test results data 270 is used to stored test results and the type of test, and test type data 280 is used to store details of the test performed such as a test name and test description. For example, the test results table may store the CFU count for a plate counting test, and the test type table may store details of the plate counting test (e.g. spread plate method, and method of counting CFUs).

The spatio-temporal infection database may be stored in a relational database, although a non-relational database including NoSQL database and flat files could be used. The spatio-temporal infection database may hosted in the cloud, or hosted at a specific location, for example on servers located at a Phage manufacturing center, or at one or more treatment locations. As outlined above, the data may be collected continuously, on demand, or periodically using a scheduled task to make periodic queries of other database systems, such as separate databases storing patient records, testing records, sequencing results, etc. These source databases may be located at treatment and testing sites or be remotely located (but operatively connected to) treatment and testing sites (e.g. a hospital server room). Thus, the spatio-temporal infection database may be generated by collecting data from multiple systems and databases at multiple locations. Data may be encrypted during transfer between systems to protect confidentiality.

The spatio-temporal infection database 200 is used to enable the identification of one or more phage suitable for inclusion in a phage formulation for treating a patient at a treatment location and/or a contaminated surface at a location. This may be performed, for example, by using a set of queries to collect or extract specific data coupled with analysis algorithms, including machine learning algorithms, constructed to process the collected data to identify to one or more infections associated with a treatment location during a historical time period. The analysis is performed to enable a phage formulation is to be matched to a particular treatment location, based on factors such as frequency of infections (a temporal trend), geographic clustering of infections (a spatial or geographic trend), and/or phage usage data (indicating which phage are being used effectively at a location). The data may also be filtered based on a resistance status, such as a multiple drug resistant (MDR) status, of a bacteria associated with an infection. For example, the resistance status may be used so that only MDR infections, or likely MDR infections are analysed. The analysis method may include generating a score based on various predictive factors, and the weighting factors may be used to emphasize (i.e. place more weight) on certain factors or values. For example more weight may be placed on recent infections, effective phage, or MDR infections. In one aspect the analysis step is an epidemiological analysis focused on identifying a temporal clustering of infections/contamination at a hospital (or other treatment location), which is then used to tune or adjust the inventory of a hospital's phage library, so that suitable quantities of phage are on hand to be used as first line treatments. Further, the analysis allows detection of changes such as development of resistance to a particular phage so the phage therapy/decontamination can be adjusted to suit the changing conditions at a particular location.

The analysis is performed over a historical time period, which is a time period ending at a recent (past) time period, including ending at the time of running a query that extracts data from the spatio-temporal infection database 200. This may be a fixed time period ending on the date or time of the newest record, or the time of running the query, or the midnight of the previous day. Alternatively, a historical time period could be defined by selecting a starting date which may give rise to a variable length time period (between successive queries). For example, the starting point could be the first day of the previous month and ending on the date of the latest record or time of running the query. Alternatively, a user could specify start and end dates. The duration of the historical time period may be the one week, one month, three months, six months, one year, or some part thereof. In cases where the analysis algorithm places greater weight on recently observed infections the time frame can be much longer—such as back to the start of record collection.

The analysis may be repeated periodically, such as daily, weekly, or monthly; or on an ad-hoc basis, for example, in response to an observed increase in presented cases at a treatment location. In one embodiment the analysis is created as a scheduled task and run at a scheduled time, or inserted into a queue of jobs to be started at a scheduled time. For example, a batch of analysis jobs, each for a different location could be created and run overnight, so that updated phage formulations can be provided to each location by the next morning. That is the method shown in FIG. 1 may include additional steps of updating the database with additional infection/contamination-related data, and then repeating the identification step for a more recent historical time period. If there is a change to the one or more phage identified as suitable for inclusion in the phage formulation, then the generation step is again performed. Thus, it will be understood that the method may thus be repeated at regular intervals to allow generation of an updated phage formulation if the most frequent types of infection/contamination change over time. When a fixed time period is used (e.g. last 3 months), then the historical time period will effectively be a sliding time window sliding by the interval between subsequent analysis runs.

For example and in preferred embodiments, the phage formulation is updated and provided to a location at intervals of at least monthly, 3 weeks, 2 weeks, 13 days, 12 days, 11 days, 10 days, 9 days, 8 days, 7 days, 6 days, 5 days, 4 days, 3 days, 2 days or 1 day prior to administration to a patient.

The analysis performed on the data over the historical time period to identify one or more phage suitable for inclusion in a phage formulation for treating a patient and/or bacterial contamination at a treatment location. Note that this does not require specific identification of the specific bacteria causing infections/contamination in each case, only identification of trends or patterns which suggest a common source such that a phage formulation to be effective at that location can be identified. For example, phage usage data with outcome measures may avoid the need for identification of the specific bacterial source of the infection.

Various analysis approaches may be used. For example, one set of queries may identify the frequency of infections/contamination at a location. This data may be aggregated (bulk) data or it may be stratified based on bacterial data such as genus, species, or ID (i.e. a separate analysis is done per bacterial genus/species/ID). The historical time period could be divided into sub time periods, and frequencies counted in each sub time period. An upward trend, or a recent increase (for example detected using a t-test or similar test to identify a variation in excess of normal variation) may indicate the presence of an emerging bacteria, or a bacteria developing resistance to existing treatments (e.g. antibiotics or phage) at a location. Additionally, or alternatively the analysis may attempt to identify geographic clustering of infections. In this embodiment cluster analysis could be used based on calculating distance measures between infection locations. This may be at level of county/suburb, or within a particular hospital, such as cluster in a particular medical unit or ward. Distance may be geographic distance calculated using geographic coordinates (e.g. latitude and longitude) or a modified distance scale for use within a hospital or treatment center taking into account buildings, floors, wards, medical units, air conditioning circuits, or other isolation or infection control structures. Again the data may be aggregated (bulk) data or it may be stratified based on bacterial data such as genus, species, or ID. Combined spatio-temporal analysis may also be performed.

The analysis may also take into phage usage data. For example, the analysis could look for changes in results of HRQT screening for samples from a location (or multiple locations), combined with monitoring of the vials of phage withdrawn from an onsite supply as well as any supplemental phage supplied to the location. For example, an increase in the number of HRQT tests, or an upsurge in usage may indicate an emerging bacterium. Preferably this data is combined with patient outcome data or bacterial contamination data to enable identification of phage that are no longer effective (e.g. bacteria is developing resistance). Similarly changing HRQT screening results (e.g. the number of phages detected as effective) may indicate a resistance issue or emergence of a new bacterial strain. For example, over time, the types of infections that present at a treatment location may change, thus necessitating a change in the first line phage formulation deployed to that treatment location.

Infections due to MDR bacteria are of particular concern to hospitals and treatment locations, as they can become entrenched at a location and extend patients stays and the cost of each stay. Moreover, these MDR bacterial infections can also contaminate the facility, making it difficult to prevent future infections. Thus, in one embodiment the analysis uses a resistance status to filter data, so that the analysis is restricted to infections/contaminations due to MDR bacteria, or likely MDR bacteria. Similarly the effectiveness of a phage over time may be assessed based on the number of treatments and the patient outcomes. If usage of a specific phage at a location increases or is constant, whilst outcomes decrease this may indicate the bacteria is developing resistance to the phage, necessitating a change to the phage formulation.

The above analysis may be performed using statistical data analytics methods and/or machine learning methods. For example time series analysis, cluster analysis, linear modelling, classification approaches, etc could be used. Deep learning methods could also be used if sufficient data is available.

In one embodiment the analysis comprises calculating a phage score, which estimates the likelihood of a phage being needed:

$${PhageScore}_{∅} = \{ {\sum }_{i = 1}^{number of patients} [ ( number of vials * {k}_{1} ) * ( \frac{1}{D aysSinceUse} ) * ( \frac{1}{distanc e} ) * clearance ] \} * {k}_{2} * n * 1000 \qquad Equation 1$$

$$where :$$

$$DaysSinceUse \geq 1 ;$$

$$Distance \geq 1 where 1 is the current location of interest ;$$

Clearance is binary where 1 indicates effective, and 0 indicates ineffective;

n is the number of patients treated with a particular phage identified with HRQT;

k₁ is a factor used to modify the number of vials, initially set at 0.1; and

k₂ is a factor used to modify n, initial set at 0.1.

The PhageScore is specific for each phage. Working through Equation 1, the equation sums across patients to capture a score contribution from all patients. The score is dependent on the number of vials given to a patient to indicate the total exposure to phage the patient received. The factor (1/DaysSinceUse) captures a time component. This emphasises recent treatments and down weights older treatments in order to adjust for possible selection/resistance effects. The factor (1/distance) captures the geographical aspect. We are most concerned with infections occurring at or near the treatment site. This is to account for changing bacteria populations in different wards, hospitals, or suburbs/counties and cities. So infections occurring at a city 1000 miles away should be down weighted compared to an infection in the same city or suburb as the treatment location. The clearance factor is there to downweight ineffective phage (i.e. if it didn't work previously, it's not expected to work again). The factor n is the number of times HRQT identified the current phage as the right phage to use for treatment.

The PhageScore is equally applicable to both veterinary use as well as a decontaminant. The formulation would stay the same mathematically, but the semantic meaning of n would change to the more general concept of observations (e.g. number of times a particular phage was used to decontaminate a surface or to treat livestock at a particular farm) whether it be on a surface for decontamination or in a veterinary application or a similar use.

Table 1 presents simulated data showing calculation of PhageScores:

TABLE 1
Simulated Data for calculation of PhageScores.

| | | | | Clear- | | |
|---|---|---|---|---|---|---|
| Phage | Vials | Days | Distance | ance | Contrib | |
| A | 7 | 110 | 106 | 1 | 0.060034305 | |
| A | 5 | 62 | 873 | 1 | 0.009237705 | |
| A | 13 | 87 | 393 | 1 | 0.038021702 | |
| A | 14 | 74 | 174 | 1 | 0.108729419 | |
| A | 12 | 130 | 250 | 1 | 0.036923077 | |
| A | 9 | 137 | 608 | 1 | 0.010804841 | |
| A | 2 | 190 | 84 | 1 | 0.012531328 | |
| A | 1 | 352 | 252 | 1 | 0.001127345 | |
| A | 12 | 294 | 167 | 1 | 0.024440914 | |
| A | 14 | 20 | 605 | 1 | 0.115702479 | |
| | Score | | | | | 0.41755311 |
| B | 3 | 108 | 815 | 1 | 0.003408316 | |
| B | 4 | 215 | 371 | 1 | 0.005014731 | |
| B | 9 | 177 | 162 | 1 | 0.03138732 | |
| B | 6 | 365 | 249 | 1 | 0.006601749 | |
| B | 12 | 204 | 925 | 1 | 0.0063593 | |
| B | 12 | 44 | 750 | 1 | 0.036363636 | |
| B | 2 | 220 | 63 | 1 | 0.014430014 | |
| B | 12 | 125 | 728 | 1 | 0.013186813 | |
| | Score | | | | | 0.0934015 |
| C | 10 | 204 | 345 | 0 | 0 | |
| C | 15 | 230 | 635 | 1 | 0.010270455 | |
| C | 9 | 82 | 410 | 1 | 0.02676978 | |
| C | 2 | 49 | 96 | 1 | 0.042517007 | |
| C | 11 | 251 | 483 | 1 | 0.009073437 | |
| C | 4 | 15 | 597 | 1 | 0.044667783 | |
| C | 7 | 316 | 167 | 1 | 0.01326461 | |
| | Score | | | | | 0.10259415 |
| D | 11 | 23 | 59 | 0 | 0 | |
| D | 1 | 273 | 559 | 1 | 0.000655278 | |
| D | 13 | 181 | 648 | 1 | 0.011083828 | |
| D | 12 | 242 | 814 | 1 | 0.006091742 | |
| D | 13 | 1 | 435 | 1 | 2.988505747 | |
| | Score | | | | | 1.5031683 |
| E | 13 | 129 | 893 | 1 | 0.011285016 | |
| E | 5 | 326 | 383 | 1 | 0.004004549 | |
| | Score | | | | | 0.00305791 |
| F | 9 | 327 | 232 | 1 | 0.011863334 | |
| F | 3 | 257 | 24 | 1 | 0.048638132 | |
| F | 11 | 35 | 764 | 1 | 0.041136874 | |
| F | 11 | 91 | 118 | 1 | 0.102439933 | |
| F | 12 | 347 | 700 | 0 | 0 | |
| | Score | | | | | 0.10203914 |

This indicates that for this treatment location (e.g. hospital/ward/unit), phage D is the most likely phage to treat this patient. It's dominated by the 5th patient that was treated successfully one day ago. Phage A is also a likely candidate since it's been identified often as the phage to use with 100% clearance relatively recently.

When graphed, these data show that phage having PhageScores greater than one standard derivation from the mean are preferred and should be included in the phage formulation as described herein.

Embodiments of the above method allows the use of a first line phage formulation that is precisely matched to a location based on geography and epidemiology. This allows a phage formulation to be tuned based on the conditions in the area where it's intended to operate. This enables dynamic forward deployment of phage formulations at treatment sites. That is, by ongoing data collection and analysis, a treatment can be provided, for example, on patient and/or contamination presentation that is updated over time to be precisely match with infections seen in the environment where it is used

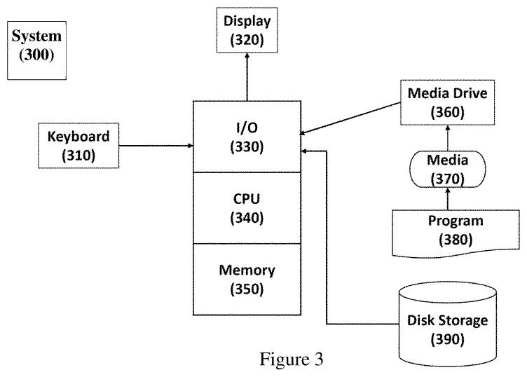

FIG. 3 depicts an exemplary computing system configured to perform any one of the computer implemented methods described herein. In this context, the computing system may include, for example, a processor, memory, storage, and input/output devices (e.g., monitor, keyboard, disk drive, Internet connection, etc.). However, the computing system may include circuitry or other specialized hardware for carrying out some or all aspects of the processes. The computer system may be a distributed system including cloud-based computing systems. In some operational settings, the computing system may be configured as a system that includes one or more units, each of which is configured to carry out some aspects of the processes either in software, hardware, or some combination thereof. For example the user interface may be provided on a desktop computer or tablet computer, whilst the training of the machine learning model and execution of a trained machine learning model may be performed on a server based system including cloud based server systems, and the user interface is be configured to communicate with such servers.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Specifically, FIG. 3 depicts computing system (300) with a number of components that may be used to perform the processes described herein. For example, an input/output (“I/O”) interface 330, one or more central processing units (“CPU”) (340), and a memory section (350). The I/O interface (330) is connected to input and output devices such as a display (320), a keyboard (310), a disk storage unit (390), and a media drive unit (360). The media drive unit (360) can read/write a computer-readable medium (370), which can contain programs (380) and/or data. The I/O interface may comprise a network interface and/or communications module for communicating with an equivalent communications module in another device using a predefined communications protocol (e.g. Bluetooth, Zigbee, IEEE 802.15, IEEE 802.11, TCP/IP, UDP, etc).

Machine learning based approaches may be implemented using machine learning libraries/packages such as SciKit-Learn, Tensorflow, and PyTorch, Turi Create, etc. These typically implement a plurality of different classifiers such as a Boosted Trees Classifier, Random Forest Classifier, Decision Tree Classifier, Support Vector Machine (SVM) Classifier, Logistic Classifier, etc. These can each be tested, and the best performing classifier selected. A computer program may be written, for example, in a general-purpose programming language (e.g., Pascal, C, C++, Java, Python, JSON, etc.) or some specialized application-specific language to provide a user interface, call the machine learning library, and export results.

A non-transitory computer-program product or storage medium comprising computer-executable instructions for carrying out any of the methods described herein can also be generated. A non-transitory computer-readable medium can be used to store (e.g., tangibly embody) one or more computer programs for performing any one of the above-described processes by means of a computer. Further provided is a computer system comprising one or more processors, memory, and one or more programs, wherein the one or more programs are stored in the memory and configured to be executed by the one or more processors, the one or more programs including instructions for carrying out any of the methods described herein.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

1. A method of selecting a phage formulation, wherein said method comprises:
(a) storing bacterial infection/contamination data in a spatio-temporal infection database, in which the data is derived from bacterial isolates from one or more treatment locations, the database comprising at least one of the following data fields: (1) a clinical indication, (2) a bacteria identification, (3) a clinical outcome, (4) a phage resistance status, (5) a phage susceptibility profile, (6) an antibiotic susceptibility profile, and/or (7) a lab test result relating to any one of (1)-(6);
(b) identifying one or more phage suitable for inclusion in the phage formulation by analyzing the data fields of (1)-(7) in the database to identify to one or more infections associated with a treatment location during a historical time period based at least on one or more of a frequency of infections/contamination, a geographic clustering of infections/contamination, and/or phage usage data;
(c) generating a selected list of one or more phage(s) to be included in the phage formulation.

2. The method of claim 1, wherein the data field comprising the bacteria identification is defined by genus, species, strain, sequence, and/or NCBI tax ID.

3. The method of either claim 1 or 2, further comprising updating the database with additional infection-related data, and repeating the identification step for a more recent historical time period and repeating the generating step if there is a change to the one or more phage identified as suitable for inclusion in the therapeutic phage formulation.

4. The method of any of the preceding claims wherein the method is performed using machine learning.

5. The method of any of the preceding claims, wherein the identifying one or more phage comprises calculating a PhageScore for each phage.

6. The method of claim 5, wherein the phage identified as having a PhageScore above one standard deviation from the mean is added to the selected list.

7. The method of any of the preceding claims, wherein said method further comprises generating the phage formulation.

8. The method of any of the preceding claims, wherein the phage formulation is generated from a phage inventory management system.

9. The method of claim 8, wherein the phage inventory management system is updated with new phage having a PhageScore higher than one standard deviation from the mean.

10. A phage formulation generated by the method of any one of claims 7-9.

11. A method of treating a patient suffering from a bacterial infection, wherein said method comprises administering to the patient the phage formulation of claim 10.

12. A method of treating a surface contaminated with bacteria, wherein said method comprises treating said surface with the phage formulation of claim 10.

13. A computing apparatus comprising:
at least one memory, and
at least one processor wherein the memory comprises instructions to configure the processor to perform the method of any one of claims 1-6.

14. A non-transitory, computer program product comprising computer executable instructions for performing the method of any one of claims 1-6.
